(19) United States
(12) Patent Application Publication
(10) Pub. No.: US 2026/0038691 A1
(43) Pub. Date: Feb. 5, 2026

(54) Health Index Determination And Fleet Monitoring For An Electrical Apparatus

(71) Applicant: Eaton Intelligent Power Limited, Dublin (IE)

(72) Inventors: Bibhudatta Patnaik, Bhubaneswar (IN); Mugdha Vyankatesh Limaye, Nigdi Pune (IN); Prasad Arvind Venikar, Pune (IN); Travis Vernon Spoone, Greenwood, SC (US); Mark Andre Faulkner, Greenwood, SC (US); Antonio Romero Oruga, Greenwood, SC (US)

(21) Appl. No.: 19/241,863

(22) Filed: Jun. 18, 2025

(30) Foreign Application Priority Data
Jun. 27, 2024 (IN) 202411049406

(51) Int. Cl. G16H 50/30

(52) U.S. Cl. CPC G16H 50/30

(57) ABSTRACT

An electrical apparatus health monitoring system includes: a health index determination module configured to: access a rank for each of a plurality of sub-parameters; determine a relative importance of each sub-parameter based on the rank of the sub-parameter, the rank of at least one other sub-parameter, and a pre-determined scale; determine a weight for each sub-parameter based on the relative importance of the sub-parameter; and determine a health index for the electrical apparatus based on the weights of the sub-parameters and scores associated with the sub-parameters; and a visualization module configured to: present the health index and the weight for each sub-parameter.

### CROSS-REFERENCE TO RELATED APPLICATION

This application claims priority to Indian Patent Application number 202411049406, filed Jun. 27, 2024 and titled HEALTH INDEX DETERMINATION AND FLEET MONITORING FOR AN ELECTRICAL APPARATUS, which is incorporated herein by reference in its entirety.

### TECHNICAL FIELD

This disclosure relates to determining a health index for an electrical apparatus and fleet monitoring.

### BACKGROUND

An electrical apparatus, such as transformer, may be used as part of an electrical system that distributes time-varying or alternating current (AC) electrical power. The electrical system may include other electrical assets, such as, for example, voltage regulators, inductors, transmission lines, and switches.

### SUMMARY

In one aspect, an electrical apparatus health monitoring system includes: a health index determination module configured to: access a rank for each of a plurality of sub-parameters; determine a relative importance of each sub-parameter based on the rank of the sub-parameter, the rank of at least one other sub-parameter, and a pre-determined scale; determine a weight for each sub-parameter based on the relative importance of the sub-parameter; and determine a health index for the electrical apparatus based on the weights of the sub-parameters and scores associated with the sub-parameters; and a visualization module configured to: present the health index and the weight for each sub-parameter.

Implementations may include one or more of the following features.

Each sub-parameter may be part of one of a plurality of parameter groups, and the health index determination module may be further configured to: determine a relative importance of each parameter group; and determine a weight for each parameter's groups based on the relative importance of the parameter group. The health index may be determined based on the weights the sub-parameters, the scores for the sub-parameters, the weights of the parameter groups, and scores associated with the parameter groups. At least one of the sub-parameters may have a score based on an output of a model.

The pre-determined scale may be non-linear.

The rank of the sub-parameter may indicate an assumed influence of the sub-parameter on the health index, and the scale is non-linear.

The health index determination module may be further configured to: determine whether any sub-parameters are not associated with one of the scores; and if any of the sub-parameters are not associated with one of the scores: determine a second weight for each sub-parameter that is associated with one of the scores; and determine a second health index for the electrical apparatus based on the second weight and the scores. The second weight may be determined based on a pair-wise comparison of the sub-parameters that are associated with one of the scores.

The pair-wise comparison may include a pair-wise comparison of the rank of the sub-parameters. In another aspect, a method includes: determining a rank for each of a plurality of sub-parameters of an electrical apparatus; determining a relative importance of each sub-parameter based on its rank, the rank of at least one other sub-parameter, and a pre-determined scale; determining a weight for each sub-parameter based on the relative importance of the sub-parameter; and determining a health index for the electrical apparatus based on the weights of the sub-parameters and a score associated with each sub-parameter.

Implementations may include one or more of the following features.

The method also may include: assigning each sub-parameter to one of a plurality of parameter groups; ranking the parameter groups; determining a relative importance of each parameter group based on the rank of the parameter group, the rank of at least one other parameter group, and the pre-determined scale; and determining a weight for each parameter group based on the relative importance of the parameter group, where the health index for the electrical apparatus is determined based on the weights of the sub-parameters, the score associated with each sub-parameter, the weights of the parameter groups, and a score associated with each parameter group. The method also may include visually presenting the health index, the sub-parameter weights, and the parameter group weights. The method also may include: determining whether any of the sub-parameters is not associated with a score; determining whether any of the parameter groups is not associated with a score; and determining a second health index for electrical apparatus based on the weights and scores of only the parameter groups and sub-parameters that are associated with a score.

The method also may include determining a second weight for each parameter group and each sub-parameter associated with a score, and the second health index may be determined based on the second weights and scores of only the parameter groups and sub-parameters that are associated with a score.

The method also may include visually presenting the second health index and the second weights.

In another aspect, a fleet monitoring system includes: a fleet monitoring module configured to: access one or more operational parameter scores from at least two electrical apparatuses in a fleet of electrical apparatuses; determine a relative importance of the at least two electrical apparatuses based on the one or more operational parameter scores and one or more weights, each operational parameter score and each weight corresponding to an operational parameter; and a fleet visualization module configured to: present the relative importance and a health index of the at least two electrical apparatuses.

Implementations may include one or more of the following features.

The fleet monitoring module may be further configured to access the health index of the at least two electrical apparatuses.

The operational parameter may include one or more of a location of the electrical apparatus, a type of the electrical apparatus, a cost metric of the electrical apparatus, and a utility of the electrical apparatus. The utility of the electrical apparatus may be based on a type of load connected to the electrical apparatus, and the type of load may include one or more: a municipal load, a residential load, an industrial load, a critical infrastructure load, or a retail load.

Implementations of any of the techniques described herein may be a system, a method, or executable instructions stored on a machine-readable medium. The details of one or more implementations are set forth in the accompanying drawings and the description below. Other features will be apparent from the description and drawings, and from the claims.

### DRAWING DESCRIPTION

FIG. 1 is a block diagram of an electrical power distribution system that includes an electrical asset and a monitoring system.

FIG. 2 shows an example of another power distribution system that includes an electrical asset and a monitoring system.

FIG. 3 is a flowchart of an example of a process for determining the health index of an electrical asset.

FIG. 4 shows an example of a metric model.

FIG. 5 is an example of a visual interface.

FIG. 6 is a block diagram of a system that includes a monitoring system and a fleet.

FIG. 7 is a flow chart of an example of a process for a fleet-level analysis.

FIG. 8 is an example of a visualization tool for identifying electrical apparatuses for prioritized repair or maintenance.

FIGS. 9A-9C show examples of collected data.

### DETAILED DESCRIPTION

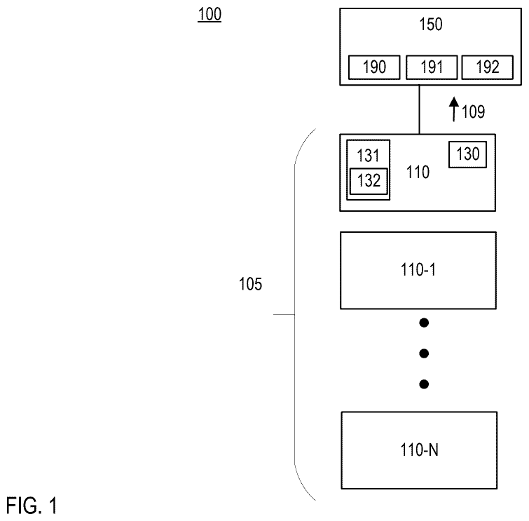

FIG. 1 is a block diagram of an electrical power distribution system 100 that includes an electrical asset 110 and a monitoring system 150. The monitoring system 150 includes a health index module 190 that determines a health index (HI) of the electrical asset 110 based on ranked parameters 131 and sub-parameters 132.

The electrical asset 110 is any type of device or machine that uses electricity. For example, the electrical asset 110 may be a transformer, a circuit breaker, a motor, a voltage regulator, or a switchgear, just to name a few. The electrical asset 110 is associated with metrics 130, which are based on data 109. The data 109 includes any data related to the electrical asset 110. The data 109 may include, for example, on-line data collected during operation of the electrical asset 110, off-line data collected while the electrical asset 110 is not in operation, modeled data, simulated data, maintenance data, information provided by the manufacturer of the electrical asset 110 (such as nameplate information), test data, and environmental data. The sub-parameters 132 are derived from the metrics 130, and the parameters 131 are groups of the sub-parameters 132.

At least some of the data 109 varies over time. The monitoring system 150 monitors and/or collects the data 109 over time and determines the HI at different points in time. By monitoring the HI over time, the monitoring system 150 can detect a decrease or reduction in the HI of the electrical asset 110. A reduction in the HI of the electrical asset 110 is an early warning sign of possible failure of the electrical asset 110. By detecting a reduction in the HI of the electrical asset 110, the monitoring system 150 may be used to capture maintenance issues and possible failures at an incipient stage, thereby reducing maintenance costs and system downtime.

Traditional approaches to determining HI for an electrical asset may include a weighted scoring technique, with the weights for the various factors or parameters used in determining the HI being provided by a human expert or technician. However, these manually provided weights are subjective and can vary among different experts or technicians. The subjective nature of the manually provided weights may lead to inaccuracies in the HI value, may mask specific failure modes due to inaccurate weights, and/or may require a relatively high number of parameters to determine an HI value to within a desired accuracy.

On the other hand, the monitoring system 150 calculates the weights based on rankings of the parameters 131 and sub-parameters 132. The rankings may be provided by a human expert or technician or may be generated through a machine learning or statistical process. The rankings are easier to understand than the weights, and, as a result, the rankings are likely to be consistent, even when provided manually by a human expert or technician. As a result, the HI of determined by the monitoring system 150 is more accurate than the HI determined by the traditional approach.

The monitoring system 150 also includes a failure analysis module 191 to facilitate identification of one or ones of the parameters 131 and/or sub-parameters 132 are the biggest contributors to a reduction in the HI. The failure analysis module 191 allows rapid and accurate identification of failure points in the electrical asset 110. The identification of specific failure points allows maintenance of the electrical asset 110 to be targeted such that the maintenance may be scheduled in a quicker, less expensive, and more efficient manner. Furthermore, the monitoring system 150 includes a fleet analysis module 192 that monitors a fleet 105 that includes the electrical asset 110 in addition other electrical assets 110-1 to 110-N.

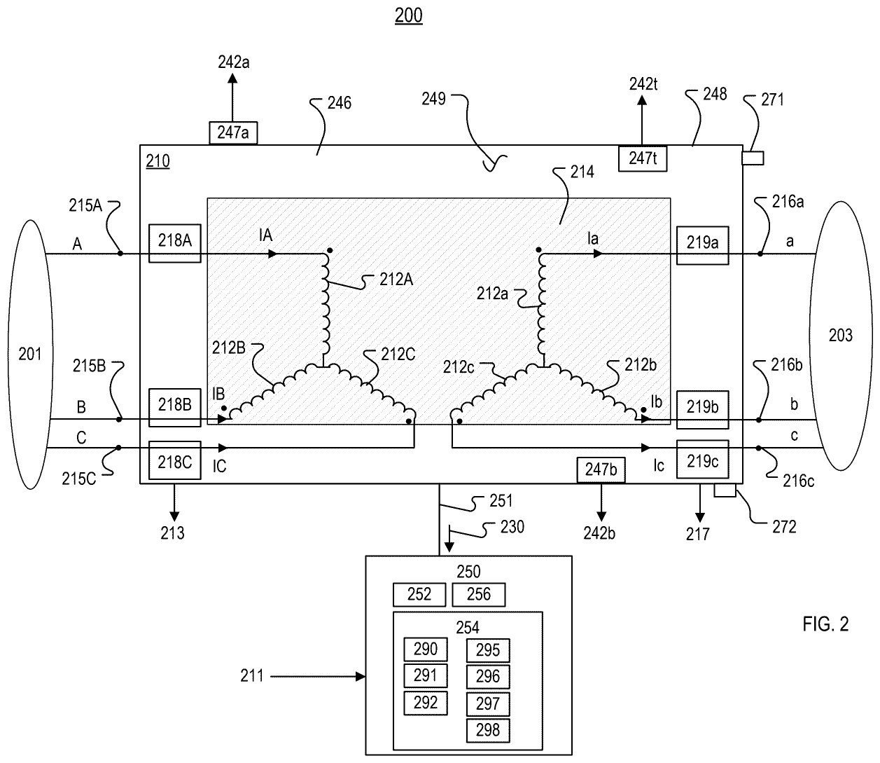

As discussed above, the electrical asset 110 may be a transformer. FIG. 2 shows an example of a power distribution system 200 that includes an electrical asset 210 (a transformer 210). The transformer 210 is a three-phase, wye-wye connected transformer that is cooled with a fluid 246, such as, for example, a synthetic or natural oil. Other configurations of the transformer 210 are possible. For example, the transformer 210 may be configured as a delta-wye transformer.

The transformer 210 is coupled to a monitoring system 250 via a connection 251. The connection 251 is any type of connection that can send data, signals, and/or commands between the transformer 210 and the monitoring system 250. The connection 251 may be, for example, an electrical cable. The monitoring system 250 may be integrated into the transformer 210 such that the monitoring system 250 and the transformer 210 are a single device or package. In some implementations, the monitoring system 250 is separate from the transformer 210. Moreover, the monitoring system 250 may be remote from the transformer 210. For example, the monitoring system 250 and transformer 210 may be separated by kilometers or meters but coupled by the connection 251.

The transformer 210 includes a housing 248 that defines an interior region 249. The interior region 249 contains the fluid 246. The transformer 210 also includes a fluid inlet 271 and a fluid outlet 272, both of which are in fluid communication with the interior region 249. The fluid 246 is intentionally introduced into the interior region 249 through the fluid inlet 271 and is intentionally removed from the interior region 249 through the fluid outlet 272.

The transformer 210 includes a thermal sensors 247t, 247b in the interior region 249. The thermal sensors 247t and 247b may be any type of thermal sensor, such as, for example, a thermocouple. The thermal sensor 247t produces a top fluid temperature indication 242t, which is an indication of the temperature of the fluid 246 at or near the inlet 271. The thermal sensor 247b produces a bottom fluid temperature indication 242b, which is an indication of the temperature of the fluid 246 at or near the fluid outlet 272.

A thermal sensor 247a is positioned to measure the ambient temperature in the environment that is exterior to the interior region 249. For example, the thermal sensor 247a may be mounted on the housing 248 or next to the exterior of the housing 248. In some implementations, the thermal sensor 247a is placed in the vicinity of the housing 248. For example, the thermal sensor 247a may be positioned one (1) meter or more from the exterior of the housing 248. The thermal sensor 247a produces an ambient temperature indication 242b, which is an indication of the temperature of the environment that surrounds the transformer 210. The thermal sensor 247a may be any kind of sensor that is capable of measuring temperature. For example, the thermal sensor 247a may be a thermocouple or a thermometer. In some implementations, the thermal sensor 247a is part of a weather station that produces meteorological data in addition to providing temperature data.

The transformer 210 includes two windings per phase in the interior region 249, as follows: a primary winding 212A and a secondary winding 212a in the A phase, a primary winding 212B and a secondary winding 212b in the B phase, and a primary winding 212C and a secondary winding 212c in the C phase. The transformer 210 also includes electrical insulation 214 (show in gray diagonal striped shading) that protects the primary and secondary windings. The electrical asset 210 has first nodes 215A, 215B, 215C and second nodes 216a, 216b, 216c.

The first nodes 215A, 215A, 215C are electrically connected to phases A, B, C of an AC power grid 201. The AC power grid 201 distributes AC current that has a fundamental frequency. The second nodes 216a, 216b, 216c are connected to phases a, b, c of a load 203. The AC power grid 201 is a three-phase power grid that operates at a fundamental frequency of, for example, 50 or 60 Hertz (Hz). The power grid 201 includes devices, systems, and components that transfer, distribute, generate, and/or absorb electricity. For example, the power grid 101 may include, without limitation, generators, power plants, electrical substations, transformers, renewable energy sources, transmission lines, reclosers and switchgear, fuses, surge arrestors, combinations of such devices, and any other device used to transfer or distribute electricity. The power grid 201 may be low-voltage (for example, up to 1 kilovolt (kV)), medium-voltage or distribution voltage (for example, between 1 kV and 35 kV), or high-voltage (for example, 35 kV and greater). The power grid 201 may include more than one sub-grid or portion.

The load 203 may be any device that uses, transfers, or distributes electricity in a residential, industrial, or commercial setting, and the load 203 may include more than one device. For example, the load 203 may be a motor, an uninterruptable power supply, or a lighting system. The load 203 may be a device that connects the transformer 210 to another portion of the power grid 201. For example, the load 203 may be a recloser or switchgear, another transformer, or a point of common coupling (PCC) that provides an AC bus for more than one discrete load. The load 203 may include one or more distributed energy resource (DER).

During operational use of the transformer 210, primary AC current IA, IB, IC flows in each respective first node 215A, 215B, 215C. A secondary AC current Ia, Ib, Ic flows from each respective second node 216a, 216b, 216c. The transformer 210 may be used to increase or decrease the amplitude of the secondary currents and voltages relative to the primary currents and voltages. When the number of turns in the primary winding 212A, 212B, 212C is greater than the number of turns in the respective secondary winding 212a, 212b, 212c, the amplitude of the secondary current Ia, Ib, Ic is greater than the amplitude of the respective primary current IA, IB, IC. When the number of turns in the primary winding 212A, 212B, 212C is less than the number of turns in the respective secondary winding 212a, 212b, 212c, the amplitude of the secondary current Ia, Ib, Ic is smaller than the amplitude of the respective primary current IA, IB, IC.

The transformer 210 also includes sensors 218A, 218B, 218C that measure one or more electrical properties at the first nodes 215A, 215B, 215C and sensors 219a, 219b, 219c that measure one or more electrical properties at the second nodes 216a, 216b, 216c. For example, each of the sensors 218A, 218B, 218C, 219a, 219b, 219c may measure current, voltage, and/or power at the respective nodes 215A, 215B, 215C, 216a, 216b, 216c. The sensors 218A, 218B, 218C, 219a, 219b, 219c may be any kind of electrical sensor, for example, current transformers (CTs), Rogowski coils, power meters, and/or potential transformers (PT).

The sensors 218A, 218B, 218C produce an indication 213, and the sensors 219a, 219b, 219c produce an indication 217. The indications 213 and 217 include data that represent measured values. For example, the indications 213 and 217 may include sets of numerical values that are each associated with a time stamp, where each set includes three measured values that represent an instantaneous value of an electrical property at one of the first nodes or one of the second nodes. Although the indications 213 and 217 are shown in the example of FIG. 2, other implementations are possible. For example, in some implementations, each sensor 218A, 218B, 218C, 219a, 219b, 219c produces a separate indication.

The transformer 210 is associated with metrics 230. The metrics 230 include any measurable or quantifiable property related to the transformer 210. The metrics 230 may include data measured during use of the transformer 210, such as the indications 213 and 217, the top fluid temperature indication 242t, the bottom fluid temperature indication 242b, and the ambient temperature indication 242a. The metrics 230 may include other data measured during use of the transformer 210. For example, the metrics 230 may include a measurement of an internal pressure, and/or a level of the fluid 246. The metrics 230 that include data measured during operation of the transformer 210 are provided to the monitoring system 250.

The metrics 230 may include data other than data measured during use of the transformer 210. For example, the metrics 230 may include data that is derived from data measured during use of the transformer 210. For example, the metrics 230 may include test data that is not necessarily obtained during operation of the transformer 210. Examples of test data for the transformer 210 include dissolved gas analysis (DGA), tests for total gas pressure, and testing for furanic compounds (FURAN testing). The metrics 230 also may include outputs of models and/or simulations. The metrics 230 also may include operational information and data, such as an indication of when the transformer 210 was first operated, when the transformer 210 was manufactured, and nameplate information associated with the transformer 210. The metrics 230 also may include maintenance data such as a historical record of previous faults that have occurred in the transformer 210 and/or a historical record of repairs. Furthermore, the metrics 230 may include observations of the transformer 210, such as a visible condition of the transformer 210 as compared to established criteria. Additional information may be included in the metrics 230. For example, the metrics 230 may include cost information including, for example, maintenance cost, replacement cost, and estimated cost associated with failure of the transformer 210.

The monitoring system 250 includes an electronic processing module 252, an electronic storage 254, and an input/output (I/O) interface 256. The electronic processing module 252 includes one or more electronic processors, each of which may be any type of electronic processor and may or may not include a general purpose central processing unit (CPU), a graphics processing unit (GPU), a microcontroller, a field-programmable gate array (FPGA), Complex Programmable Logic Device (CPLD), and/or an application-specific integrated circuit (ASIC).

The electronic storage 254 is any type of electronic memory that is capable of storing data and instructions in the form of computer programs or software, and the electronic storage 254 may include volatile and/or non-volatile components. The electronic storage 254 and the processing module 252 are coupled such that the processing module 252 can access or read data from and write data to the electronic storage 254.

The electronic storage 254 stores information about the transformer 210 and may store at least some of the metrics 230. For example, the electronic storage 254 may store nameplate information 211. The nameplate information 211 may include, for example, the rated temperature of the insulation 214 (or the critical hotspot temperature limit); the rated load of the transformer 210; the number of turns on the windings winding 212A, 212B, 212C, 212a, 212b, 212c; a voltage and/or current rating of the transformer 210; a heat capacity of the material of the windings 212A, 212B, 212C, 212a, 212b, 212c; an identifier or flag that indicates the electrical configuration of the transformer 210; and/or an arrangement of the bushings on the transformer 210. The critical hotspot temperature limit is the highest temperature that the insulation 214 is designed to tolerate. The nameplate information 211 is loaded onto the electronic storage 254 via the I/O interface 256. For example, an operator may enter the nameplate information 211 while the transformer 210 is in the field. In another example, the manufacturer of the transformer 210 may add or edit the nameplate information 211 via the I/O interface 256.

The electronic storage 254 may store other of the metrics 230. For example, the electronic storage 254 may store test results, historical fault data, maintenance data, and operational information and data. Furthermore, the electronic storage 254 may store data that is based on measurements taken during operation of the transformer 210. The metrics 230 may be stored in a database, a collection of data structures, or a lookup table.

The electronic storage 254 also stores executable instructions that cause the processing module 252 to perform various operations. The executable instructions may be stored in the form of, for example, a computer program, logic, or software. For example, the electronic storage 254 includes executable instructions that implement various condition modeling modules. The condition modeling modules shown FIG. 2 are an electrical model 295, a thermal model 296, a fluid leak detection model 297, and a gas model output 298.

The electrical model 295 outputs an electrical apparatus health indicator, which provides information about the health of the transformer 210 and is one of the metrics 230. The electrical apparatus health indicator 258 may be a binary value that indicates whether or not an electrical fault (for example, a short) is present in any of the windings 212A, 212B, 212C, 212a, 212b, 212c. In some implementations, the electrical model 295 uses the indications 213 and/or 217 to determine whether an unbalanced condition exists, and the value of the electrical apparatus health indicator depends on whether the unbalanced condition exists.

The thermal model 296 determines an estimate of one or more thermal parameters of the transformer 210. For example, the thermal model 296 may estimate the winding hotspot temperature (OH), which is an estimate of the highest temperature or maximum temperature of the winding 212. The hotspot temperature (OH) at a particular time may be predicted or estimated using various equations that are set forth in the IEEE C57.91 Standard, Annex G.

The fluid leak detection model 297 determines a temperature error and uses the temperature error to determine whether or not a fluid leak condition exists in the transformer 210. The temperature error may be determined based on the measured top fluid temperature indication 242t and an estimate of the top fluid temperature determined by the model 297.

The electronic storage 254 may store additional or fewer models. Moreover, the features and operation of the electrical model 295, the thermal model 296, the fluid leak detection model 297, and the gas model output 298 discussed above are provided as examples and any of the models 295, 296, 297, 298 may be implemented in other ways. Regardless of their specific implementation, the output of the models 295, 296, 297, 298 are part of the metrics 230.

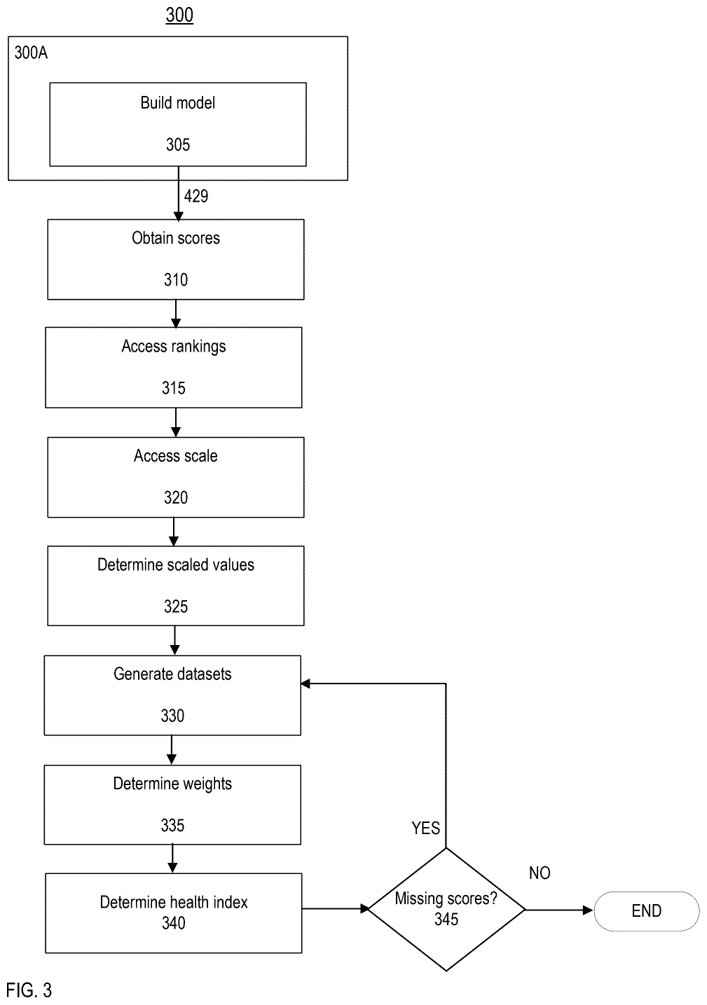

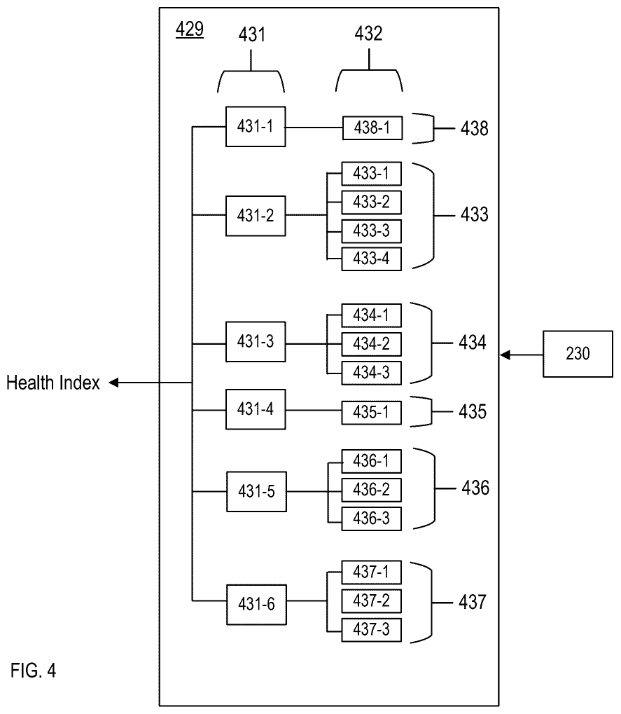

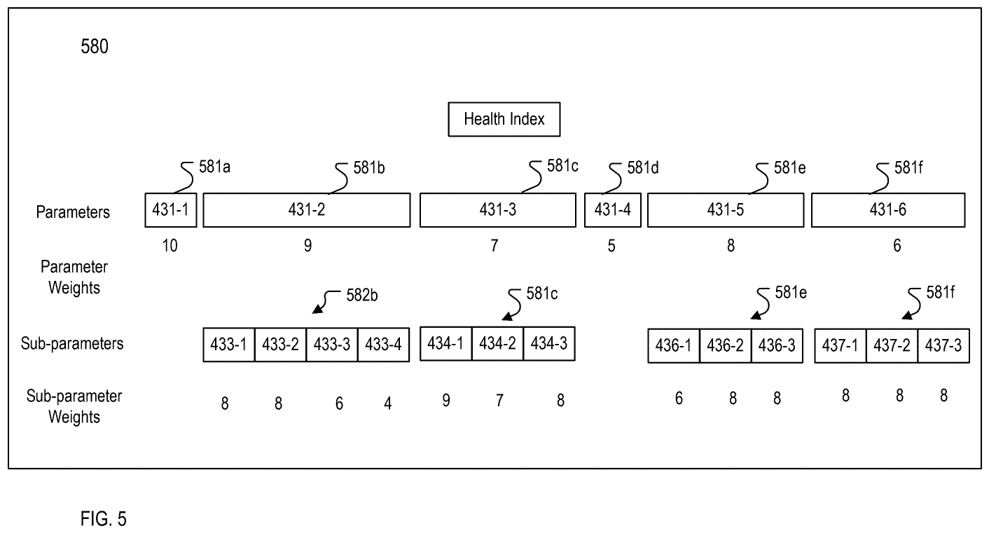

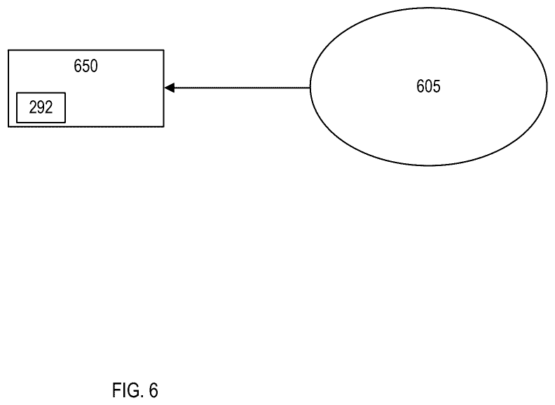

The electronic storage 254 includes executable instructions that implement a health index (HI) module 290. The HI module 290 determines the HI of the transformer 210. FIGS. 3 and 4 discuss the HI module 290 in more detail. The executable instructions also include a failure analysis module 291 that analyzes causes of reduced HI and presents a visual interface at the I/O interface 256. FIG. 5 discusses an example of the visual interface. Additionally, the electronic storage 254 includes executable instructions that implement a fleet monitoring module 292. The fleet monitoring module 292 is discussed with respect to FIGS. 6-8.

The I/O interface 256 is any interface that allows a human operator, another electronic device, and/or an autonomous process to interact with the monitoring system 250. The I/O interface 256 may include, for example, a display (such as a liquid crystal display (LCD)), a keyboard, audio input and/or output (such as speakers and/or a microphone), visual output (such as lights, light emitting diodes (LED)) that are in addition to or instead of the display, serial or parallel port, a Universal Serial Bus (USB) connection, and/or any type of network interface, such as, for example, Ethernet. The I/O interface 256 also may allow communication without physical contact through, for example, an IEEE 802.11, Bluetooth, or a near-field communication (NFC) connection. The monitoring system 250 may be, for example, operated, configured, modified, or updated through the I/O interface 256.

The I/O interface 256 also may allow the monitoring system 250 to communicate with systems external to and remote from the monitoring system 250 and the transformer 210. For example, the I/O interface 256 may include a communications interface that allows communication between the monitoring system 250 and a remote station (not shown), or between the monitoring system 250 and a separate electrical apparatus (such as another transformer) using, for example, the Supervisory Control and Data Acquisition (SCADA) protocol or another services protocol, such as Secure Shell (SSH) or the Hypertext Transfer Protocol (HTTP). The remote station may be any type of station through which an operator is able to communicate with the monitoring system 250 without making physical contact with the monitoring system 250. For example, the remote station may be a computer-based work station, a smart phone, tablet, or a laptop computer that connects to the monitoring system 250 via a services protocol or a telephone system, or a remote control that connects to the monitoring system 250 via a radio-frequency signal. The monitoring system 250 may communicate information to an external device through the I/O interface 256.

FIG. 3 is a flowchart of a process 300 for determining the HI of an electrical asset, such as the electrical asset 110 or the transformer 210. The process 300 is discussed with the transformer 210 to provide an example. However, the process 300 may be used to determine the HI of any electrical asset. In the example discussed below, the process 300 is implemented as a collection of executable instructions that form the health index determination module 290 and are stored on the electronic storage 254. The process 300 is performed by the monitoring system 250.

The process 300 includes a sub-processes 300A in which a metric model 429 is built (305). FIG. 4 shows an example of the metric model 429. The sub-process 300A is performed prior to the remaining portions of the process 300 but is not necessarily performed every time the process 300 is performed. In other words, the metric model 429 may be generated or built once and used many times. The metric model 429 is built by identifying metrics 230 or information derived from the metrics 230 to be sub-parameters 432. The identification may be a manual process that is performed by an expert or experienced technician. In some implementations, the identification of the metrics 230 is based on pre-determined criteria or rules that are applied by a human expert or technician or by an electronic processor.

The sub-parameters 432 are grouped into sub-parameter groups 433, 434, 435, 436, 437, 438, each of which is associated with a parameter category 431. The sub-parameters 432 may be grouped manually (for example, by an expert or a technician) or based on a pre-determined set of rules. Sub-parameters within a category have a common feature and/or attribute. A specific example of the parameter categories 431 and the sub-parameters 432 grouped into each category 431 is shown in Table 1.

TABLE 1

| Priority | Parameter Categories | Sub-parameters |
|---|---|---|
| (439) | (431) | (432) |
| Health | Load History | Overloads (438-1) |
| Index | (431-1) | |
| | Condition Monitoring | Electrical model output (433-1) |
| | Analysis and Modeling | Thermal model output (433-2) |
| | (431-2) | Oil leakage model output (433-3) |
| | | Gas model output (433-4) |
| | Service Cost Data | Maintenance cost (434-1) |
| | (431-3) | Expense associated with failure (434-2) |
| | | Asset importance (434-3) |
| | Transformer condition | Qualitative assessment based |
| | (431-4) | on observation (435-1) |
| | | Maintenance data (435-2) |
| | Other Operation Data | Number of start-up and |
| | (431-5) | shut-down actions (436-1) |
| | | Age (436-2) |
| | | Number of fault events (436-3) |
| | Other Condition | Pressure measurement (437-1) |
| | Measurement Data | Oil level (437-2) |
| | (431-6) | Flooded vault (437-3) |

The parameter categories 431 and sub-parameters 432 in Table 1 are provided as an example. Other parameter categories 431 and/or sub-parameters 432 may be used in the model 429.

After defining the model 429, scores are obtained for the sub-parameters 432 and the parameters 431 (310). The scores are based on the data that make up metrics 230. The scoring strategy varies among the sub-parameters 432 and parameters 431. For example, the overloads 431-1, maintenance cost 434-1, and expense associated with failure 434-2 have scores that are determined directly from the metric 230 associated with each of these sub-parameters, with higher values of the associated metric 230 resulting in a lower score for the sub-parameter.

To provide a more specific example, the score of the overloads 438-1 sub-parameter is based on measurements (for example, the measured current or voltage indications 213 and 217) and a count of the number of times that an overload condition (an over-voltage or over-current condition) occurred during a finite period of time. The score assigned to overloads 438-1 is inversely related to the count of overloads during the time period such that the more times an overload condition occurred during a time period, the lower the score of the overloads 438-1 sub-parameter. The score assigned to maintenance cost 434-1 and expense associated with failure 434-2 are inversely related to the respective costs of maintenance and failure. A transformer that is expensive to maintain has a low score for the maintenance cost 434-1 sub-parameter and a transformer that is inexpensive to maintain has a high score for the maintenance cost 434-1 sub-parameters. In another example, for electrical model output 433-1 sub-parameter, the underlying metric 230 is the output of the electrical model 295 and the score of the electrical model output 433-1 sub-parameter is based on the output of the electrical model 295.

Other scoring strategies are used. Additionally, some of the sub-parameters, such as the qualitative assessment 435-1 and maintenance data 435-2 are based on metrics 230 that include user input and user observations that are standardized relative to pre-defined criteria. Table 2 provides an example of a scoring strategy for the maintenance data 435-2.

| Score | Scoring Criteria |
|---|---|
| A | Maintained fewer than 3 times in the past 2 years |
| | OR Maintenance increased <10% over the last 5 years |
| B | Maintained more than 3 times in the past 2 years |
| | AND Maintenance increased >10% over the last 5 years |
| C | Maintained more than 5 times in the past 2 years |
| | AND Maintenance increased >30% over the last 5 years |
| D | Maintained more than 10 times in the past 2 years |
| | AND Maintenance increased >50% over the last 5 years |
| E | Maintained more than 15 times in the past 2 years |
| | AND Maintenance increased >50% over the last 5 years |

Each character score A to E is assigned a numerical value. For example, the character scores A, B, C, D, E may be assigned respective numerical values 5, 4, 3, 2, 1. Furthermore, the scores for the sub-parameters 432 may be normalized or standardized to be between a specific range (for example, between 0 and 1 or between 0 and 4, with zero being the lowest score).

Rankings of the sub-parameters 432 and the parameter categories 431 are accessed (315). The rankings include criticality rankings and reliability rankings. The criticality rankings indicate the relative influence of a sub-parameter or parameter on HI. Sub-parameters and parameters that have more influence on the HI have a higher ranking, and sub-parameters that have a weaker influence on the HI have a lower ranking. Reliability rankings indicate the relative data quality of metrics 230 associated with the various sub-parameters and parameters. A lower reliability ranking indicates a low confidence in the underlying metric 230 and a higher reliability ranking indicates a greater confidence in the underlying metric 230.

The criticality and reliability rankings may be provided by a service technician. In some implementations, the criticality and/or reliability rankings are pre-determined or pre-defined and are stored on the electronic storage 254. The criticality and reliability rankings may be in any form. For example, the rankings may be a linear scale that starts at 1 with other ranking values incremented by 1 to the maximum ranking value. The maximum ranking value depends on how many rankings are included. Table 3 shows an example of a linear ranking with 9 different possible rankings.

TABLE 3

| Relative Importance | Ranking Value | Scale |
|---|---|---|
| Equal | 1 | 1.00 |
| Weak | 2 | 1.25 |
| Moderate | 3 | 1.50 |
| Moderate Plus | 4 | 1.75 |
| Strong | 5 | 2.50 |
| Strong Plus | 6 | 4.00 |
| Very Strong | 7 | 5.50 |
| Very Very Strong | 8 | 7.00 |
| Extreme | 9 | 9.00 |

In the example shown in Table 3, a ranking of 1 corresponds to “equal.” Sub-parameters and parameters having a criticality ranking of “equal” are equally likely to affect or not affect the HI value. In other words, sub-parameters and parameters having the criticality ranking value of 1 are the least important as far as determining HI. Sub-parameters and parameters that have a baseline reliability are “equal” and have a reliability ranking of 1. Sub-parameters and parameters that have scores based on data known or expected to have greater availability and/or accuracy have a higher ranking. If all of the scores are based on data that is equally likely to be reliable, the ranking value of all of the sub-parameters and parameters is set to “equal” with a reliability ranking value of 1.

The highest ranking value in the example shown in Table 3 is 9. Sub-parameters and parameters having a criticality ranking of “extreme” have a criticality ranking value of 9 and exert the most influence on the HI. Sub-parameters or parameters having a reliability ranking of “extreme” have a reliability ranking value 9. The ranking may take other forms. For example, the ranking may be based on a color scale, with each ranking assigned to a different color and each color corresponding to a numerical ranking value.

Regardless of the form of the ranking, the ranking is in a simple and easy-to-use format. Although the ranking for the various sub-parameters 432 and parameters 431 is provided by the service personnel, service personnel do not provide the weights of the sub-parameters 432 and the parameter categories 431. Instead, the weights are calculated based on the rankings as discussed below. This is in contrast to legacy health index (HI) calculations that rely on subjective weights provided by the service personnel. As compared to weights, rankings are more intuitive and more likely to remain consistent among different service personnel and among different scenarios. Thus, the approach used in the process 300 (and implemented in the monitoring system 250) produces a more accurate health index (HI) than the legacy approaches in which the weights are provided instead of being calculated. For example, the approach used in the process 300 is less prone to errors and/or inaccuracies in the HI calculation that can arise from using subjective weights that are provided by service personnel.

The weights of the sub-parameters 432 and parameters 431 are determined by applying a scale to the ranked sub-parameters to determine scaled sub-parameters and then generating datasets that are based on pairwise comparisons of the scaled sub-parameters and scaled parameters, as discussed below. A scale that relates the criticality rankings and the reliability rankings to a numerical value is accessed (320). Table 3 shows an example of a scale that relates each ranking value to a numerical scaled value. In the example shown in Table 3, the relationship between the ranking values and the scale values is non-linear and the scale values increase non-linearly from the “equal” ranking to the “extreme” ranking. The non-linear relationship between the sub-parameters may provide more realistic results as compared to using a linear scale.

A scaled criticality value is determined for each sub-parameter based on its criticality ranking to produce scaled criticality sub-parameters (325). For example, a sub-parameter that has a criticality ranking of 1 is equally likely to influence or not influence the value of HI and is assigned a scaled value of 1. A sub-parameter that has a criticality ranking of 2 weakly influences the HI value is assigned a scaled value of 1.25.

A dataset is generated for each sub-parameter group 433, 434, 435, 436, 437, 438 and for the parameters 431 (330). Each dataset is a matrix and may be referred to as the comparison matrix (C). Equation 1 shows the determination of a comparison matrix (C):

$$C = \vert \begin{matrix}{c}_{11} = 1 & {c}_{12} = 1 / {c}_{21} & {c}_{13} = 1 / {c}_{31} & … & {c}_{1 n} \\ {c}_{21} & {c}_{22} = 1 & {c}_{23} = 1 / {c}_{32} & … & {c}_{2 n} \\ {c}_{31} & {c}_{32} & {c}_{33} = 1 & … & {c}_{3 n} \\ ⋮ & ⋮ & ⋮ & ⋮ & ⋮ \\ {c}_{m 1} & {c}_{m 2} & {c}_{m 3} & … & {c}_{mn} = 1\end{matrix} \vert , \qquad ( Equation 1 )$$

where m indexes the rows of the matrix (C), n indexes the columns of the matrix (C), and each value c represents the relative importance of two scaled sub-parameters or two scaled parameters. The values of c along the diagonal of the comparison matrix (C) represent the comparison of the criticality ranking of a sub-parameter or parameter with itself and thus have a value of 1. A comparison matrix (C) is determined for each sub-parameter group 433, 434, 435, 436, 437, 438. An additional comparison matrix is determined for the parameters 431. Table 4 provides an example comparison matrix (C) for the sub-parameter group 434 with the scale shown in Table 3. The values in the comparison matrix shown in Table 4 are determined by a pairwise comparison based on Equation 1.

TABLE 4

| | Criticality Ranking | | |
|---|---|---|---|
| | 4 | 1 | 1 |
| | Scaled Value | | |
| | 1.75 | 1 | 1 |
| | Maintenance | Expense associated | Asset |
| | cost | with failure | importance |
| Maintenance cost | 1 | 1/1.75 = 0.571 | 1/1.75 = 0.571 |
| (434-1) | | | |
| Expense | 1/0.571 = 1.75 | 1 | 1/1 = 1 |
| associated with | | | |
| failure (434-2) | | | |
| Asset importance | 1/0.571 = 1.75 | 1/1 = 1 | 1 |
| (434-3) | | | |

A comparison matrix (C) is determined for each sub-parameter group 433, 436, and 437 in the same manner. The sub-parameter groups 435 and 438 include only one sub-parameter and a comparison matrix is not generated for groups 435 and 438. A comparison matrix (C) is also determined for the parameters 431 in the same manner using the criticality ranking of each parameter 431. Table 5 shows an example criticality matrix (C) for the parameters 431 determined based on Equation 1 and the criticality rankings shown in Table 5.

TABLE 5

| | | | | | | | 5 |
|---|---|---|---|---|---|---|---|
| | | 2 | | | | 5 | Other |
| | | Condition | 3 | 1 | 6 | Other | Condition |
| | | Monitoring | Service | Load | Transformer | Operation | Measurement |
| Ranking | | Analysis | Cost Data | History | condition | Data | Data |
| 2 | Condition Monitoring Analysis | 1.00 | 1.25 | 0.80 | 2.50 | 1.75 | 1.75 |
| 3 | Service Cost Data | 0.80 | 1.00 | 0.67 | 1.75 | 1.50 | 1.50 |
| 1 | Load History | 1.25 | 1.50 | 1.00 | 4.00 | 2.50 | 2.50 |
| 6 | Transformer condition | 0.40 | 0.57 | 0.25 | 1.00 | 0.80 | 0.80 |
| 5 | Other Operation Data | 0.57 | 0.67 | 0.40 | 1.25 | 1.00 | 1.00 |
| 5 | Other Condition Measurement Data | 0.57 | 0.67 | 0.40 | 1.25 | 1.00 | 1.00 |

Additionally, a reliability comparison matrix (C) is determined for each group of sub-parameters 433, 434 435, 436, and 437 and for the parameters 431. The reliability comparison matrices are determined based on Equation (1) in the same manner as the criticality matrices are determined. Table 6 shows an example reliability matrix for the parameters 431 determined based on Equation 1 and the reliability rankings shown in Table 6.

TABLE 6

| | | | | | | | 1 |
|---|---|---|---|---|---|---|---|
| | | 1 | 1 | | | 1 | Other |
| | | Condition | Service | 1 | 1 | Other | Condition |
| | | Monitoring | Cost | Load | Transformer | Operation | Measurement |
| Ranking | | Analysis | Data | History | condition | Data | Data |
| 1 | Condition Monitoring Analysis | 1 | 1 | 1 | 1 | 1 | 1 |
| 1 | Service Cost Data | 1 | 1 | 1 | 1 | 1 | 1 |
| 1 | Load History | 1 | 1 | 1 | 1 | 1 | 1 |
| 1 | Transformer condition | 1 | 1 | 1 | 1 | 1 | 1 |
| 1 | Other Operation Data | 1 | 1 | 1 | 1 | 1 | 1 |
| 1 | Other Condition Measurement Data | 1 | 1 | 1 | 1 | 1 | 1 |

The weight (w) of for each sub-parameter 432 and parameter 431 is determined (335). Equation (2) may be used to determine an intermediate weight of each sub-parameter 432 and parameter 431:

$${w}_{i} = \frac{{\sum }_{m = 1}^{M} \frac{{C}_{im}}{{\sum }_{n = 1}^{N} Cnm}}{M} , \qquad Equation ( 2 )$$

where i is an integer greater than or equal to 1 that indexes the sub-parameters 432 and the parameters 431, wᵢ is the intermediate weight of the ith sub-parameter 432 or parameter 431, C is the comparison matrix for the ith sub-parameter 432 or parameter 431, M is the total number of rows in the comparison matrix for the ith sub-parameter 432 or parameter 431, m is an integer that indexes the rows of the comparison matrix, N is the total number of columns in the comparison matrix, and n is an integer that indexes the columns of the comparison matrix. As shown in Equation (2), the intermediate weight (w) is determined from a ratio of an individual component value (Cim) compared to the total component value. Equation (2) is used to determine an intermediate weight (w) for each sub-parameter 432 and parameter 431 is determined from the criticality matrices and from the reliability matrices such that each sub-parameter 432 and parameter 431 has an intermediate criticality weight (wi,CR) and an intermediate reliability weight (Wi,R).

The final weight (Wi,final) for each sub-parameter 432 and parameter 431 is calculated based on Equation (3):

$${W}_{iFinal} = {W}_{i , R} {W}_{R} + {W}_{i , CR} {W}_{CR} , \qquad Equation ( 3 )$$

where i is an integer that indexes the sub-parameters 432 and the parameter 431, and WR and WCR are numerical values that assign relative importance to the intermediate reliability weight (WiR) and the intermediate criticality weight (WiCR), respectively. The values of WR and WCR may be user-defined inputs or may be pre-defined and stored on the electronic storage 254.

The health index (HI) is determined (340). The determination of the health index (HI) is based on Equations (4) and (5). Equation (4) is used to determine an intermediate sub-parameter score (sP) for each group of sub-parameters 433, 434, 435, 436, 437, 438:

$$sP = \frac{{\sum }_{i = 1}^{n} {S}_{sPi} {W}_{sPi}}{{Sg}_{max} {\sum }_{i = 1}^{n} {W}_{sPi}} , \qquad Equation ( 4 )$$

where n is the number of sub-parameters in the group, i is an integer that indexes n, wsPi is the final weight of the ith sub-parameter in the group, Sspi is the score of the ith sub-parameter in the group, and Sgmax is the maximum score of the sub-parameters in the group. The scores of the sub-parameters 432 are based on the underlying metric or metrics 230 associated with the sub-parameter. For example, the sub-parameters 433-1, 433-2, 433-3, 433-4 of the group 433 may have scores and final weights as shown in Table 6.

TABLE 6

| Sub-parameter | Score | Final weight |
|---|---|---|
| Electrical model output (433-1) | 5 | 0.3 |
| Thermal model output (433-2) | 4 | 0.3 |
| Oil leakage model output (433-3) | 4 | 0.2 |
| Gas model output (433-4) | 1 | 0.2 |

In this example, n is four (4) and the sub-parameter score (sP) for the group 433 is 0.74. The sub-parameter health index (sP) is determined for the other groups of sub-parameters using Equation (4). After the sub-parameter score (sP) is determined for all of the groups of sub-parameters, the health index (HI) is determined using Equation (5):

$$HI = \frac{{\sum }_{i = 1}^{m} {S}_{Pi} {W}_{Pi}}{{S}_{max} {\sum }_{i = 1}^{m} {W}_{Pi}} , \qquad Equation ( 5 )$$

where m is the number of parameters, i is an integer that indexes m, wPi is the final weight (wi final) of the ith parameter, Spi is the score of the ith sub-parameter group associated with the ith parameter as determined in Equation (4), and Smax is the maximum score of the sub-parameter groups 433, 434, 435, 436, 437, 438 as determined in Equation (4). The process 300 may be performed again at a later time to determine additional HI values or the process 300 may end after determining a single HI value.

In some implementations, a recursive calculation is performed (345) if any of the sub-parameters 432 used in the determination of the HI lack score values (Ssp(i) in Equation (4)), the process 300 returns to (330) to re-generate the datasets without the sub-parameters 432 that lack scores. The datasets are re-generated in the same manner as discussed above except any sub-parameters 432 that do not have associated scores are not considered in the calculation. For example, if the sub-parameter maintenance cost 434-1 lacked a score, the criticality matrix (C) for the sub-parameter group 434 shown in Table 4 would be recalculated without the maintenance cost 434-1 using Equation (1). In this example, the re-calculated criticality matrix (C) would be a 2×2 matrix instead of the 3×3 matrix shown in Table 4. The process 300 proceeds to determine the weights for the other sub-parameters in the group 434 at (335) and the HI at (340). The recursive calculation (345) helps in addressing the challenges of data unavailability dynamically and without having to rely on multiple sets of pre-determined and manually provided weights to cover specific situations.

The process 300 may be performed without the recursive calculation (345). In these implementations, the process 300 ends after determining the HI at (340). Moreover, although the recursive calculation (345) is shown as occurring after the HI is determined at (340), this is not necessarily the case. For example, in some implementations, missing scores are assessed at (310) and any sub-parameters 432 that lack scores are not included in the initial HI calculation. Moreover, any of the value calculated by the process 300 may be stored on the electronic storage 254 for subsequent analysis or processing and/or presented at the I/O interface 256. For example, the weights determined in (335) and the HI determined in (340) may be stored on the electronic storage 254 and/or presented at the I/O interface 256.

Referring to FIG. 5, an example health visualization 580 is shown. The health visualization is produced by the failure analysis module 291. The health visualization 580 displays a fault tree analysis (FTA) that allows a user to quickly pinpoint the sub-parameters 432 and parameters 431 that contribute the most to the HI. The health visualization 580 includes the HI determined by the process 300. The health visualization 580 also includes labels 581a to 581f, each of which is associated with one of the parameters 431. The health visualization 580 also includes labels 582b, 582c, 582e, 582f, each of which is associated with a group of the sub-parameters 432. The labels 581a to 581f and the labels 582b, 582c, 582e, 582f are shown with numerical data that identifies the parameter or sub-parameter but may be implemented as textual labels.

The health visualization 580 also includes the weights associated with each parameter 431 and sub-parameter 432. The weights are calculated in (335) as discussed above with respect to FIG. 3. The weights may be normalized prior to display in the health visualization 580. For example, if the weights determined at (335) are between 0 and 1, the weights may be multiplied by 10 to produce a clearer display.

The calculated weight associated with a particular parameter or sub-parameter is shown directly under the label corresponding to the parameter or sub-parameter. For example, the weight associated with the parameter 431-1 (the load history) is 10 and the weight associated with the sub-parameter 433-1 (electrical model output) is 8.

The health visualization 580 provides a visual assessment of the impact of the various parameters 431 and sub-parameters 432 on the HI. In the example shown, the parameters load history (431-1) and condition modeling (431-2) have the highest impact on the HI. The viewer can also readily assess from the visualization 580 that, within the condition modeling (431-2) parameter, the sub-parameters electrical model 433-1 and thermal model 433-2 have the greatest impact on the HI. In response to reviewing the health visualization 580, the viewer can schedule maintenance or an in-person review of the transformer 210 that focuses on the loading of the transformer 210 and checking the fluid 246. This enables maintenance to be more effectively managed, for example, because the appropriate service personnel and tools can be selected based on the review of the visualization 580. Moreover, the service can be scheduled early to repair or adjust a component or sub-system of the transformer that is performing sub-optimally before that component or sub-system causes issues throughout the transformer.

FIG. 6 is a block diagram of a system 600 that includes a monitoring system 650 and a fleet 605. The monitoring system 650 includes the fleet monitoring module 292. The fleet 605 includes at least two electrical apparatuses and may include tens, hundreds, or thousands of electrical apparatuses. The monitoring system 650 may be configured to access and/or receive HI values from the electrical apparatuses in the fleet 605. In some implementations, the monitoring system 650 is similar to the monitoring system 250 and receives metrics 230 from all of the monitored assets and determined HI values for the electrical apparatuses in the fleet based on the process 300. The fleet 605 of apparatuses may include only transformers. However, in some implementations, the fleet 605 includes additional and/or other equipment. For example, the fleet 605 may include transformers as well as other monitored equipment (such as, for example, circuit breakers, motors, reclosers, and switchgear). In some implementations, the fleet 605 lacks transformers and includes only monitored equipment other than transformers. In other words, the fleet 605 may have any type of monitored electrical assets or equipment.

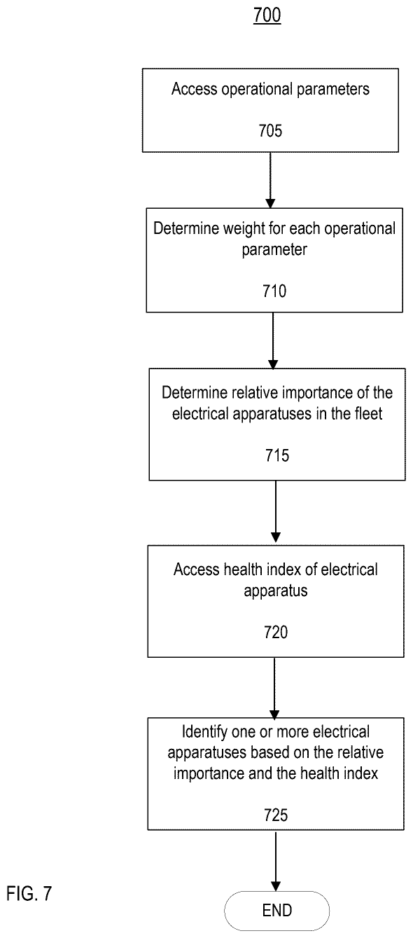

FIG. 7 is a flow chart of a process 700 for a fleet-level analysis. The process 700 may be used to schedule and/or prioritize maintenance or repairs within the fleet 605. The process 700 may be implemented by the fleet monitoring module 292.

Operational parameters of the electrical apparatuses in the fleet 605 are accessed (705). The operational parameters include any information related to the operation and use of the electrical apparatuses in the fleet 605. Examples of operational parameters include: the location of each electrical apparatus, the type of electrical apparatus, the end user of the electrical apparatus, and the overall cost of the electrical apparatus.

Each operational parameter is associated with categories or classes, allowing the electrical apparatuses in the fleet 605 to be characterized based on the operational parameters. The classes of location may be based on population density, for example, large city, medium city, small city, rural area. The type of electrical apparatus may include, for example, power transformer, distribution transformer, auxiliary transformer, and network transformer. The utility of the electrical apparatus may include classes related to the type of load typically driven by the electrical apparatus. For example, the utility classes may include medical facilities, government facilities, and residential facilities. The overall cost may be a numerical value or score that accounts for factors such as, for example, asset replacement cost, downtime impact, and maintenance cost.

The various operational parameters are associated with numerical values based on a pre-defined scale or rule. For example, the location parameter of large city may have a value of 4, medium city a value of 3, small city a value of 2, and rural area a value of 1. Each electrical apparatus in the fleet 605 has an assigned value (or score) for each operational parameter. The score for each operational parameter may be assigned by a manager of the fleet 605 and stored in a database or lookup table in the monitoring system 650.

A weight for each operational parameter is determined (710). The weight may be determined using a pair-wise comparison approach by such as discussed above with respect to Equation (1), or the weights may be provided by the operator or manager of the fleet 605. In some implementations, the weights are pre-defined for the fleet 605 and remain unchanged during management of the fleet 605. In implementations that use the pair-wise comparison approach, the HI for each apparatus in the fleet 605 as determined by Equation (5) may be used as the score (or one of the scores) associated with that apparatus. The weight of the apparatuses in the fleet 605 may be assigned based on, for example, the type of apparatus or the use of the apparatus.

The relative importance of the electrical apparatuses in the fleet 605 is determined (715). The relative importance of an individual electrical apparatus in the fleet 605 is determined from a weighted sum of the operational parameter scores associated with the electrical apparatus. For the four example operational parameters discussed above, the relative importance of the ith electrical apparatus in the fleet 605 is determined using Equation (6):

$${rel\_imp}_{i} = w\_loc ( s\_loc ( i ) ) + w\_typ ( s\_typ ( i ) ) + w\_utl ( s\_ult ( i ) ) + w\_cst ( s\_cst ( i ) ) , \qquad Equation ( 6 )$$

where i is a integer number that indexes the electrical apparatuses in the fleet 605, w_loc is the weight of the location operational parameter, w_typ is the weight of the type operational parameter, w_utl is the weight of the utility operational parameter, w_cst is the weight of the overall cost operational parameter, s_loc(i) is the score of the location operational parameter of the ith electrical apparatus, s_type(i) is the score for the type operational parameter of the ith electrical apparatus, s_utl(i) is the score for the utility operational parameter of the ith electrical apparatus, and s_cst(i) is the score for the overall cost operational parameter of the ith electrical apparatus.

The operational parameters shown in Equation (6) are examples, and more or fewer operational parameters may be used. For example, the relative importance may be determined without considering the overall cost operational parameter. In this example, Equation (6) would include only three terms. Moreover, other operational parameters may be used to determine the relative importance of the electrical apparatuses in the fleet 605. For example, a score for the expected lifetime operational parameter may be associated with each electrical apparatus in the fleet 605. In this implementation, the process 700 includes determining a weight for the lifetime operational parameter and the weighted sum determined by Equation (6) includes the weighted expected lifetime score for the ith electrical apparatus. In another example, the HI value of the apparatus may be used as a score.

The HI of the electrical apparatuses in the fleet 605 are accessed (720). The HI of the electrical apparatuses in the fleet 605 may be determined based on the process 300 discussed above or the HI may be determined in another manner and provided to the fleet monitoring system 650. One or more electrical apparatuses are identified based on the HI and the relative importance (725). For example, a schedule for repairing or maintaining the electrical apparatuses in the fleet 605 may be determined by identifying a first group of critical electrical apparatuses that will be serviced during a first day, a second group of moderately important electrical apparatuses that will be serviced after the first group, and a third group of less important electrical apparatuses that will be serviced after the first and second groups.

The identification of the electrical apparatuses based on HI and relative importance may be mathematical and/or performed by reviewing a visual display. An example of a mathematical approach is shown in Equation (7):

$$fleet\_rank ( i ) = k \frac{( rel\_imp ( i ) )}{HI ( i )} , \qquad Equation ( 7 )$$

where rel_imp(i) is the relative importance of the ith electrical apparatus as determined by Equation (6), HI(i) is the health index of the ith electrical apparatus, k is a numerical value used to scale the fleet rank value to a whole number, and fleet_rank(i) is a numerical metric relating to the ranking of the ith electrical apparatus within the fleet 605. The HI may be determined based on the process 300 or the HI may be provided by the fleet operator. With Equation (7), the fleet_rank increases as the relative importance increases and the HI decreases. Thresholds may be applied to the fleet_rank metric to identify electrical apparatuses for prioritized repair or maintenance.

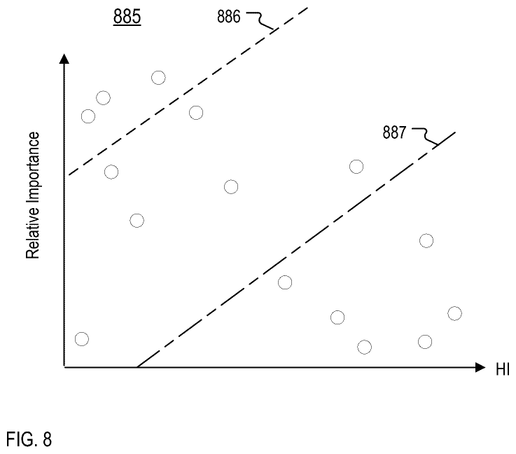

FIG. 8 is an example of a visualization tool 885 for identifying electrical apparatuses for prioritized repair or maintenance. The visualization tool 885 is a scatter plot of the relative importance (rel_imp) versus health index (HI) of the electrical apparatuses in the fleet 605. Each open circle in FIG. 8 represents one electrical apparatus. The visualization tool 885 includes a first threshold 886 (dashed line) and a second threshold 887 (dot-dash line). Electrical apparatuses that are above and to the left of the first threshold 886 have the highest relative importance and the lowest health index and are the highest priority for repair. Electrical apparatus that are below and to the right of the second threshold 887 have the lowest relative importance and the highest health index and are the lowest priority for repair. Electrical apparatuses between the first threshold 886 and the second threshold 887 have moderate importance and moderate health indexes and may or may not need to be repaired.

The thresholds 886 and 887 may be pre-defined by, for example, the manager or owner of the fleet 605. In some implementations, the visualization tool 885 is configured to allow an end user to adjust the thresholds 886 and 887.

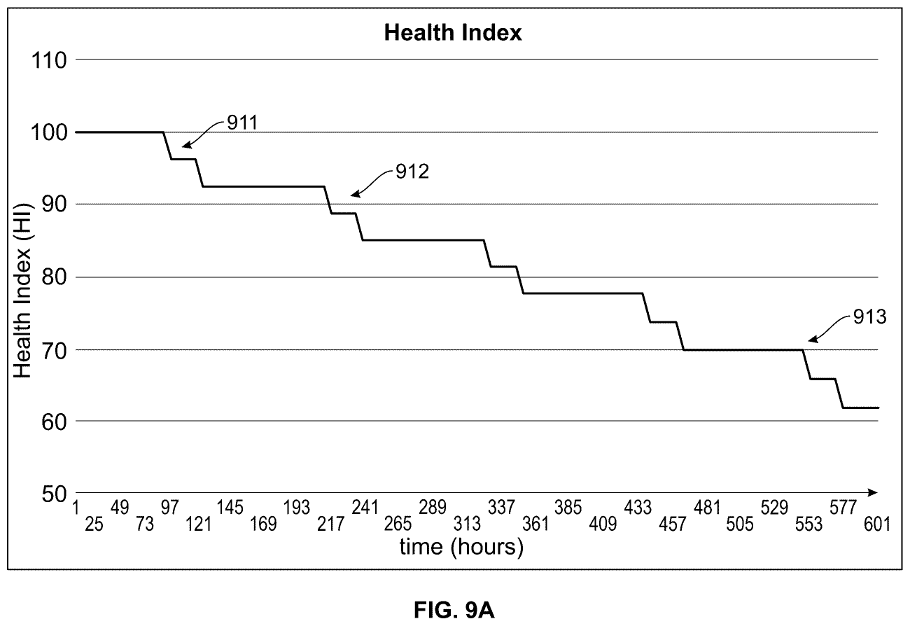

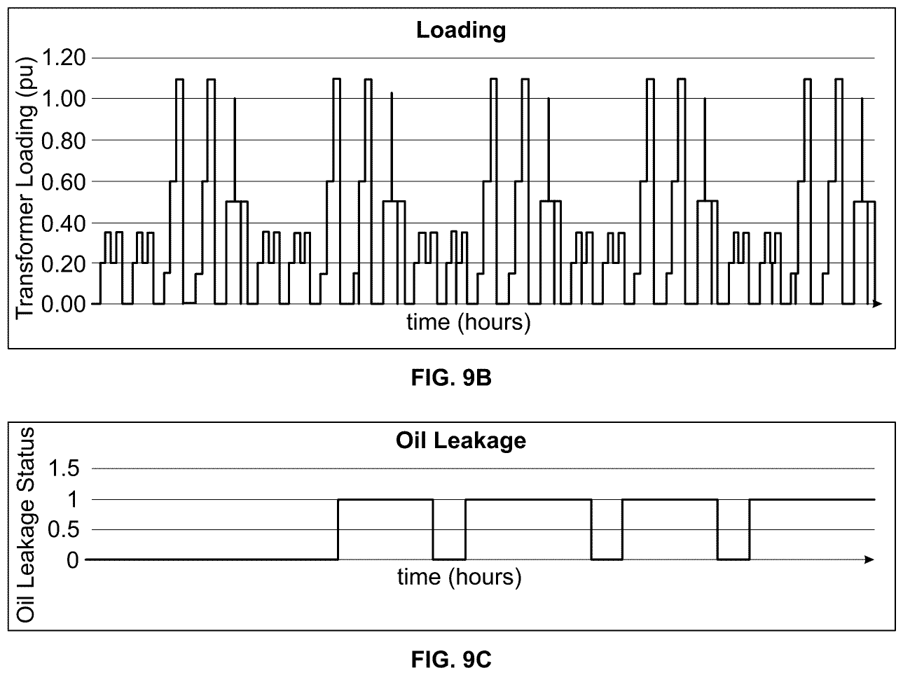

FIGS. 9A-9C show examples of data collected during a pilot implementation. FIG. 9A is a plot of health index (HI) as a function of time. The HI was calculated based on the process 300 discussed above. FIG. 9B is a plot of transformer loading as a function of time. FIG. 9C is a plot of oil leakage status as a function of time. The time scale is the same in FIGS. 9A-9C. The simulation was conducted by simulating a 250 kVA transformer. The loading levels (minute wise) of the transformer were L1 to L5 over 5 days. In the simulation, L1 was 20% loading, L2 was 38% loading, L3 was 60% loading, L4 was 100% loading, and L5 was greater than 100% loading. The oil leakage status of the transformer (day way) was 0 for no oil leakage and 1 for oil leakage. The hot spot temperature was calculated from a thermal model. The number of starts and starts were based on the starting and stopping of transformer between load level changes (for example, from L1 to L2).

As shown in FIGS. 9A-9C, the HI decreased during high loading durations and due to continuous oil leakage since the third day onwards. For example, the HI decreased at the time labeled 911 when the loading levels exceeded 100% and at the time labeled 912, when the loading levels exceeded 6100 and there was an oil leak. For the loading levels below 60%, there was no decrease in HI. Further, even during the time when loading was below 60% and there was an oil leakage (such as at the time labeled 913), there is small but consistent decrease in the Health Index. These observations are consistent with expectations and support that the HI calculation based on the process 300 produces accurate results.

These and other implementations are within the scope of the claims.

What is claimed is:

1. An electrical apparatus health monitoring system comprising:
a health index determination module configured to:
access a rank for each of a plurality of sub-parameters;
determine a relative importance of each sub-parameter based on the rank of the sub-parameter, the rank of at least one other sub-parameter, and a pre-determined scale;
determine a weight for each sub-parameter based on the relative importance of the sub-parameter; and
determine a health index for the electrical apparatus based on the weights of the sub-parameters and scores associated with the sub-parameters; and
a visualization module configured to:
present the health index and the weight for each sub-parameter.

2. The electrical apparatus health monitoring system of claim 1, wherein each sub-parameter is part of one of a plurality of parameter groups, and the health index determination module is further configured to:
determine a relative importance of each parameter group;
determine a weight for each parameter's groups based on the relative importance of the parameter group, and wherein the health index is determined based on the weights the sub-parameters, the scores for the sub-parameters, the weights of the parameter groups, and scores associated with the parameter groups.

3. The electrical apparatus health monitoring system of claim 2, wherein at least one of the sub-parameters has a score based on an output of a model.

4. The electrical apparatus health monitoring system of claim 1, wherein the pre-determined scale is non-linear.

5. The electrical apparatus health monitoring apparatus of claim 1, wherein the rank of the sub-parameter indicates an assumed influence of the sub-parameter on the health index, and the scale is non-linear.

6. The electrical apparatus health monitoring system of claim 1, wherein the health index determination module is further configured to:
determine whether any sub-parameters are not associated with one of the scores; and
if any of the sub-parameters are not associated with one of the scores:
determine a second weight for each sub-parameter that is associated with one of the scores; and
determine a second health index for the electrical apparatus based on the second weight and the scores.

7. The electrical apparatus health monitoring system of claim 6, wherein the second weight is determined based on a pair-wise comparison of the sub-parameters that are associated with one of the scores.

8. The electrical apparatus health monitoring system of claim 7, wherein the pair-wise comparison comprises a pair-wise comparison of the rank of the sub-parameters.

9. A method comprising:
determining a rank for each of a plurality of sub-parameters of an electrical apparatus;
determining a relative importance of each sub-parameter based on its rank, the rank of at least one other sub-parameter, and a pre-determined scale;
determining a weight for each sub-parameter based on the relative importance of the sub-parameter; and
determining a health index for the electrical apparatus based on the weights of the sub-parameters and a score associated with each sub-parameter.

10. The method of claim 9, further comprising:
assigning each sub-parameter to one of a plurality of parameter groups;
ranking the parameter groups;
determining a relative importance of each parameter group based on the rank of the parameter group, the rank of at least one other parameter group, and the pre-determined scale; and
determining a weight for each parameter group based on the relative importance of the parameter group; and wherein the health index for the electrical apparatus is determined based on the weights of the sub-parameters, the score associated with each sub-parameter, the weights of the parameter groups, and a score associated with each parameter group.

11. The method of claim 10, further comprising:
visually presenting the health index, the sub-parameter weights, and the parameter group weights.

12. The method of claim 10, further comprising:
determining whether any of the sub-parameters is not associated with a score;
determining whether any of the parameter groups is not associated with a score; and
determining a second health index for electrical apparatus based on the weights and scores of only the parameter groups and sub-parameters that are associated with a score.

13. The method of claim 12, further comprising determining a second weight for each parameter group and each sub-parameter associated with a score, and wherein the second health index is determined based on the second weights and scores of only the parameter groups and sub-parameters that are associated with a score.

14. The method of claim 12, further comprising visually presenting the second health index and the second weights.

15. A fleet monitoring system comprising:
a fleet monitoring module configured to:
access one or more operational parameter scores from at least two electrical apparatuses in a fleet of electrical apparatuses;
determine a relative importance of the at least two electrical apparatuses based on the one or more operational parameter scores and one or more weights, each operational parameter score and each weight corresponding to an operational parameter; and
a fleet visualization module configured to:
present the relative importance and a health index of the at least two electrical apparatuses.

16. The fleet monitoring system of claim 15, wherein the fleet monitoring module is further configured to access the health index of the at least two electrical apparatuses.

17. The fleet monitoring system of claim 15, wherein the operational parameter comprises one or more of a location of the electrical apparatus, a type of the electrical apparatus, a cost metric of the electrical apparatus, and a utility of the electrical apparatus.

18. The fleet monitoring system of claim 17, wherein the utility of the electrical apparatus is based on a type of load connected to the electrical apparatus, and the type of load comprises one or more: a municipal load, a residential load, an industrial load, a critical infrastructure load, or a retail load.
